(19) United States
(12) Patent Application Publication
(10) Pub. No.: US 2021/0295964 A1
(43) Pub. Date: Sep. 23, 2021

(54) INFORMATION PROCESSING SYSTEM, INFORMATION PROCESSING METHOD, AND NON-TRANSITORY STORAGE MEDIUM

(71) Applicant: NEC Corporation, Tokyo (JP)

(72) Inventor: Yosuke KOJIMA, Tokyo (JP)

(73) Assignee: NEC Corporation, Tokyo (JP)

(21) Appl. No.: 17/196,351

(22) Filed: Mar. 9, 2021

(30) Foreign Application Priority Data
Mar. 19, 2020 (JP) 2020-048574

(51) Int. Cl. G16H 10/60

(52) U.S. Cl. CPC G16H 10/60

(57) ABSTRACT

An information processing system in the present invention includes a storage unit that stores first data including no personal information on a person and does not store second data including the personal information; a first output unit that outputs the first data to a first apparatus in response to a request from the first apparatus; and a second output unit that acquires the second data from a second apparatus and outputs, to the second apparatus, third data obtained by combining the personal information with the first data.

### INCORPORATION BY REFERENCE

This application is based upon and claims the benefit of priority from Japanese patent application No. 2020-048574, filed on Mar. 19, 2020, the disclosure of which is incorporated herein in its entirety by reference.

### TECHNICAL FIELD

The present invention relates to an information processing system, an information processing method, and a non-transitory storage medium.

### BACKGROUND ART

Japanese Patent Application Laid-open No. 2014-52822 discloses an electronic medical record screening system that, while efficiently performing screening work, by using an electronic medical record system, restricts patient information included in a screening result and thereby prevents leakage of personal information on a patient.

### SUMMARY

The system described in Japanese Patent Application Laid-open No. 2014-52822 as an example is configured such that a server storing the screening result and a server storing personal information on the patient directly communicate with each other when the personal information on the patient and the screening result are associated with each other. However, when unauthorized access to one server is made via an external network, data stored in the other server may leak at the same time, and there is room for improvement in terms of security.

Accordingly, in view of the above problem, the present invention intends to provide an information processing system, an information processing method, and a non-transitory storage medium that can prevent leakage of personal information and facilitate use of data including no personal information.

According to one example aspect of the present invention, provided is an information processing system including: a storage unit that stores first data including no personal information on a person and does not store second data including the personal information; a first output unit that outputs the first data to a first apparatus in response to a request from the first apparatus; and a second output unit that acquires the second data from a second apparatus and outputs, to the second apparatus, third data obtained by combining the personal information with the first data.

According to another example aspect of the present invention, provided is an information processing method performed by an information processing system including a storage device that stores first data including no personal information on a person and does not store second data including the personal information, and the information processing method includes steps of: outputting the first data to a first apparatus in response to a request from the first apparatus; and acquiring the second data from a second apparatus and outputting, to the second apparatus, third data obtained by combining the personal information with the first data.

According to yet another example aspect of the present invention, provided is a non-transitory storage medium in which a program is stored, the program that causes a computer having a storage device that stores first data including no personal information on a person and does not store second data including the personal information to perform: outputting the first data to a first apparatus in response to a request from the first apparatus; and acquiring the second data from a second apparatus and outputting, to the second apparatus, third data obtained by combining the personal information with the first data.

According to the present invention, an information processing system, an information processing method, and a non-transitory storage medium that can prevent leakage of personal information and facilitate use of data including no personal information can be provided.

### BRIEF DESCRIPTION OF THE DRAWINGS

FIG. 1 is a block diagram illustrating an example of an overall configuration of an information processing system in a first example embodiment.

FIG. 2 is a block diagram illustrating an example of a hardware configuration of a data management apparatus in the first example embodiment.

FIG. 3 is a sequence diagram illustrating an example of a collection process of sensor data in the first example embodiment.

FIG. 4 is a diagram illustrating an example of use history data stored by an operation terminal in the first example embodiment.

FIG. 5 is a diagram illustrating an example of sensor data stored in a sensor data DB in the first example embodiment.

FIG. 6 is a sequence diagram illustrating an example of a process of displaying personal information and sensor data in association with each other in the first example embodiment.

FIG. 7 is a diagram illustrating an example of merge data displayed on the operation terminal in the first example embodiment.

FIG. 8 is a sequence diagram illustrating an example of a reference process of sample data in the first example embodiment.

FIG. 9 is a diagram illustrating an example of sample data displayed on a user terminal in the first example embodiment.

FIG. 10 is a sequence diagram illustrating an example of a reference process of sensor data in the first example embodiment.

FIG. 11 is a block diagram illustrating an example of an overall configuration of an information processing system in a second example embodiment.

FIG. 12 is a flowchart illustrating an example of a classification process of sensor data in the second example embodiment.

FIG. 13 is a sequence diagram illustrating an example of a reference process of sensor data in the second example embodiment.

FIG. 14 is a block diagram illustrating an example of an overall configuration of an information processing apparatus in a third example embodiment.

FIG. 15 is a block diagram illustrating an example of an overall configuration of an information processing system in a modified example embodiment.

FIG. 16 is a block diagram illustrating an example of an overall configuration of an information processing system in the modified example embodiment.

### EXAMPLE EMBODIMENT

Illustrative example embodiments of the present invention will be described below with reference to the drawings. In the drawings, the same elements or corresponding elements are labeled with the same reference, and the description thereof may be omitted or simplified.

### First Example Embodiment

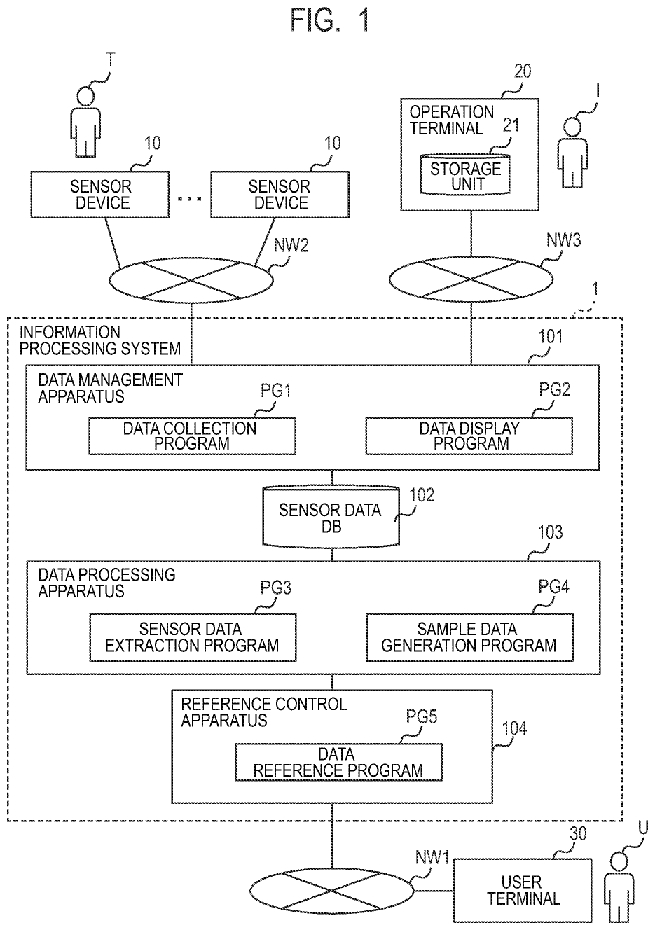

FIG. 1 is a block diagram illustrating an example of an overall configuration of an information processing system 1. The information processing system 1 of the present example embodiment is a computer system that collects and stores, in a storage device, sensor data measured in sensor devices 10 and discloses the sensor data stored in the storage device to a user terminal 30 via a network NW1 such as the Internet.

The sensor device 10 is a measurement device that outputs the sensor data obtained by measuring a subject T. The sensor device 10 is connected to a data management apparatus 101 of the information processing system 1 via a network NW2 such as a LAN or the Internet. Any type of sensor device 10 and any number of sensor devices 10 may be employed. Hereinafter, a case where two sensors, namely, an attitude sensor that measures an attitude state during the subject T being sleeping and a heart rate sensor that measures the pulse of the subject T are used as the sensor device 10 will be described as an example. In the present example embodiment, a case where the subject T is a child will be described.

An operation terminal 20 is a terminal device used by an inspector I who operates the sensor device 10 and performs collection work of sensor data for the inspector I's own operation. In the present example embodiment, the inspector I is a nurse, a nursery school teacher, or the like who manages a health condition of children. The operation terminal 20 is connected to a data management apparatus 101 of the information processing system 1 via a network NW3 such as a LAN or the Internet. The operation terminal 20 has a storage unit 21 that stores use history data of the sensor device 10 and personal information on the subject T. In the present example embodiment, the expression “personal information” is information related to any one individual and refers to information that can identify a specific individual by description or the like included in the information.

The user terminal 30 is a terminal device used by a user U when referencing sensor data disclosed by the information processing system 1. In the present example embodiment, the user U is a researcher, a medical doctor, or the like who studies information during the subject T (child) being sleeping. A specific example of the operation terminal 20 and the user terminal 30 may be a personal computer, a smartphone, a tablet terminal, or the like but not limited thereto. Note that, while the networks NW1, NW2, and NW3 may form the same wide area network, the user terminal 30 is unable to directly access the operation terminal 20.

As illustrated in FIG. 1, the information processing system 1 has the data management apparatus 101, a sensor data DB 102, a data processing apparatus 103, and a reference control apparatus 104. The respective apparatuses are server computers, for example, and are connected to each other via a network such as a LAN. Note that the data management apparatus 101 is connected to only the sensor device 10, the operation terminal 20, and the sensor data DB 102 and formed such that the data management apparatus 101 is unable to be directly accessed from the data processing apparatus 103 and the reference control apparatus 104.

The data management apparatus 101 has a data collection program PG1 and a data display program PG2. The data collection program PG1 is a program that collects and registers the sensor data measured by the sensor device 10 in the sensor data DB 102.

The data display program PG2 is a program that associates the data respectively stored in the operation terminal 20 and the sensor data DB 102 with each other and displays the associated data on the operation terminal 20 in response to a display request from the operation terminal 20.

Specifically, the data display program PG2 has a function of displaying, on the operation terminal 20, merge data (third data) obtained by combining sensor data (first data) stored in the sensor data DB 102 with data (second data) including the personal information stored in the operation terminal 20 (storage unit 21). Note that the data display program PG2 is unable to non-temporarily store the merge data in a storage medium of the data management apparatus 101.

The sensor data DB 102 is a storage device that stores the sensor data collected from the plurality of sensor devices 10 in the data management apparatus 101. That is, the sensor data DB 102 centrally stores sensor data. The sensor data stored in the sensor data DB 102 is able to be referenced and acquired from only the data management apparatus 101 and the data processing apparatus 103.

The data processing apparatus 103 is an application server, for example. The data processing apparatus 103 has a sensor data extraction program PG3 and a sample data generation program PG4. The sensor data extraction program PG3 is a program that extracts sensor data from the sensor data DB 102 in response to a request from the reference control apparatus 104. The request from the reference control apparatus 104 may be a reference request of sample data and a reference request of the sensor data (actual data).

The sample data generation program PG4 is a program that generates sample data (fourth data) by randomly acquiring, on an item basis, one value belonging to each data item of the sensor data stored in the sensor data DB 102 and combining the acquired values in response to a reference request of the sample data from the reference control apparatus 104. That is, while the sample data has the same data item as the sensor data stored in the sensor data DB 102, the respective data values are different from each other. Note that an algorithm of generating the sample data is not limited to the above.

The reference control apparatus 104 has a data reference program PG5. The data reference program PG5 is a program that displays data (sample data or sensor data) acquired from the data processing apparatus 103 on the user terminal 30 in response to a reference request from the user terminal 30.

When the reference control apparatus 104 receives a sensor data reference request from the user terminal 30, the data processing apparatus 103 (sensor data extraction program PG3) extracts the sensor data stored in the sensor data DB 102 and transmits the extracted sensor data to the reference control apparatus 104 in response to the request from the reference control apparatus 104.

When the reference control apparatus 104 receives a sample data reference request from the user terminal 30, the data processing apparatus 103 (sample data generation program PG4) generates sample data from the sensor data stored in the sensor data DB 102 and transmits the generated sample data to the reference control apparatus 104 in response to the request from the reference control apparatus 104.

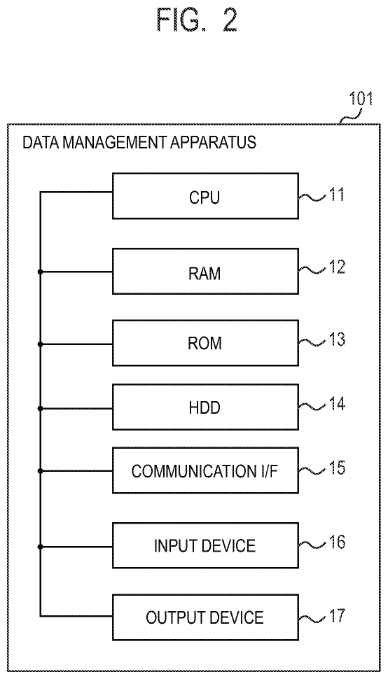

FIG. 2 is a block diagram illustrating an example of a hardware configuration of the data management apparatus 101. The data management apparatus 101 has a CPU 11, a random access memory (RAM) 12, a read only memory (ROM) 13, a hard disk drive (HDD) 14, a communication interface (I/F) 15, an input device 16, and an output device 17. The CPU 11, the RAM 12, the ROM 13, the HDD 14, the communication I/F 15, the input device 16, and the output device 17 are connected to each other via a signal line.

While components forming the data management apparatus 101 are illustrated as an integrated apparatus in FIG. 2, some of these functions may be provided by an external device. For example, the input device 16 and the output device 17 may be an external device independent of a part forming the function of a computer including the CPU 11.

The CPU 11 is a processor having a function of performing predetermined calculation in accordance with a program stored in the ROM 13, the HDD 14, or the like and also controlling each component of the data management apparatus 101. The RAM 12 is formed of a volatile storage medium and provides a temporary memory area necessary for the operation of the CPU 11. The ROM 13 is formed of a nonvolatile storage medium and stores necessary information such as a program used for the operation of the data management apparatus 101. The HDD 14 is a storage device that is formed of a nonvolatile storage medium and performs storage of a database, storage of an operation program of the data management apparatus 101, or the like.

The communication I/F 15 is a communication interface based on the specification such as Ethernet (registered trademark) or the like, which is a module used for communicating with other apparatuses.

The input device 16 is a keyboard, a pointing device, or the like and is used by the administrator of the information processing system 1 for operating the data management apparatus 101. An example of the pointing device may be a mouse, a trackball, a touch panel, a pen tablet, or the like.

The output device 17 is a liquid crystal display, an organic light emitting diode (OLED) display, a speaker, or the like and is used for presenting information for a user such as an administrator, displaying a graphical user interface (GUI), or the like. The input device 16 and the output device 17 may be integrally formed as a touch panel.

Note that the hardware configuration illustrated in FIG. 2 is an example, and a device other than the above may be added, or some of the devices may not be provided. Further, some of the devices may be replaced with another device having the same function. Furthermore, a part of the function of the present example embodiment may be provided by another device via a network, or the function of the present example embodiment may be implemented by being distributed in a plurality of devices.

Since each hardware configuration of the data processing apparatus 103, the reference control apparatus 104, the operation terminal 20, and the user terminal 30 is the same as the configuration illustrated in FIG. 2, the description thereof is omitted.

The operation in the information processing system 1 formed as described above will be described below with reference to the drawings.

(A) Collection of Sensor Data

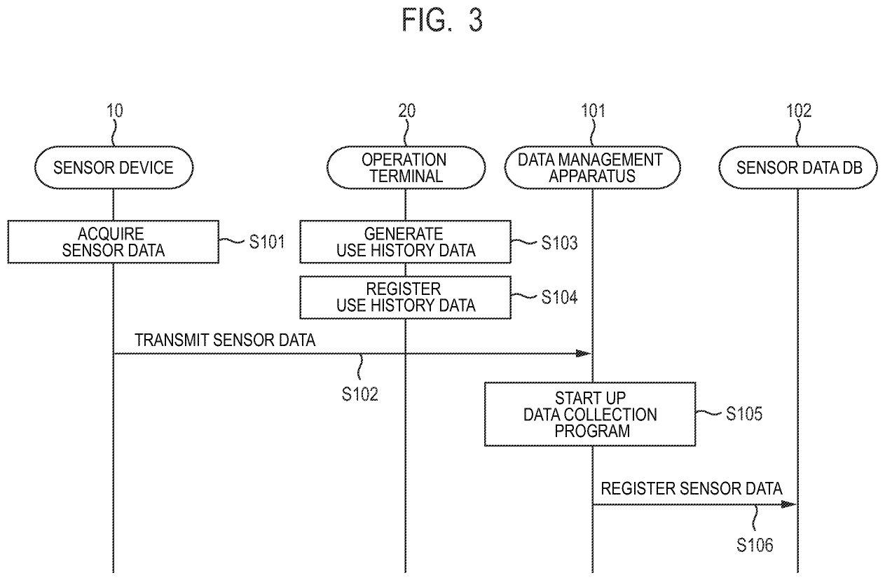

FIG. 3 is a sequence diagram illustrating an example of a collection process of sensor data. The process is performed when the inspector I uses the sensor device 10 and measures data on the subject T, for example.

First, if the sensor device 10 acquires sensor data related to the subject T (step S101), the sensor device 10 transmits the sensor data to the connected data management apparatus 101 (step S102).

At this time, the operation terminal 20 generates use history data related to use of the sensor device 10 in response to an input operation of the inspector I (step S103) in parallel with step S101 and step S102, and the use history data is registered in the storage unit 21 (step S104). The use history data includes identification data that uniquely identifies a correspondence relationship between sensor data and personal information.

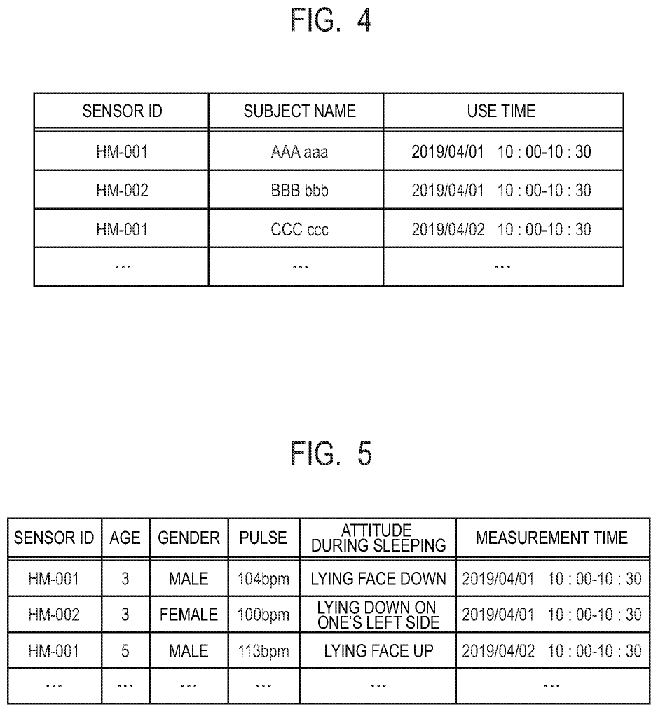

FIG. 4 is a diagram illustrating an example of the use history data stored in the operation terminal 20 (storage unit 21). Herein, a data item of the use history data may be a sensor ID, a subject name, and use time. The sensor ID is an identifier that is unique to the sensor device 10. The subject name is a name of a child who is the subject T and corresponds to personal information. The use time is the time the sensor data related to the subject T was measured by using the sensor device 10. In such a way, the inspector I registers, in the operation terminal 20, use history data indicating which sensor device 10 was used and whose sensor data was measured.

Note that the identification data includes at least two of the plurality of data items included in sensor data (first data). The data item included in the identification data is not necessarily required to be identification information that can uniquely identify the sensor data by the first item. In the present example embodiment, as identification data that uniquely identifies a correspondence relationship between sensor data and personal information, a combination of a sensor ID and use time is used. Hereinafter, a combination of a sensor ID and use time is referred to as “identification data”.

In step S105, in response to receiving the sensor data from the sensor device 10, the data management apparatus 101 starts up the data collection program PG1.

The data management apparatus 101 then registers the sensor data in the sensor data DB 102 (step S106).

FIG. 5 is a diagram illustrating an example of sensor data stored in the sensor data DB 102. Herein, the data items of the sensor data may be a sensor ID, an age, a gender, pulses, an attitude during sleeping, and measurement time. While the age and gender are attribute information on the subject T, the subject T is unable to be uniquely identified from only these pieces of information. As described above, the sensor data includes no personal information that uniquely identifies the subject T.

(B) Display of Personal Information and Sensor Data at Operation Terminal 20

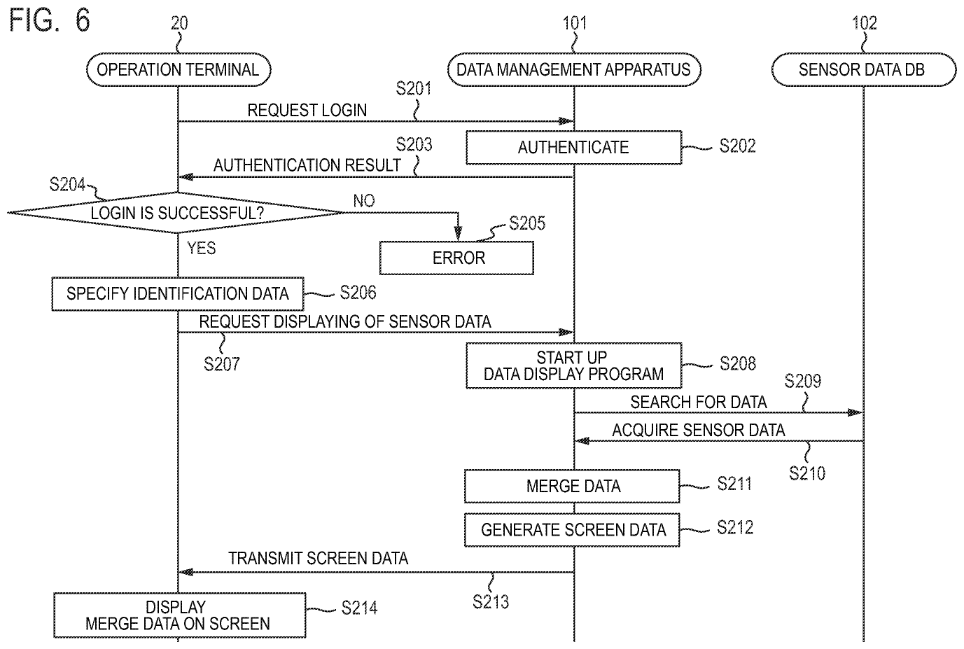

FIG. 6 is a sequence diagram illustrating an example of a process of displaying personal information and sensor data in association with each other. The process is performed when personal information and sensor data related to the desired subject T are displayed on the operation terminal 20.

First, the operation terminal 20 outputs a login request to the data management apparatus 101 (step S201). If the data management apparatus 101 performs an authentication process in response to the login request from the operation terminal 20 (step S202), the data management apparatus 101 transmits the authentication result to the operation terminal 20 (step S203).

Next, the operation terminal 20 determines whether or not the login is successful based on the authentication result in the data management apparatus 101 (step S204). Here, if the operation terminal 20 determines that the login is successful (step S204: YES), the process proceeds to step S206. On the other hand, if the operation terminal 20 determines that the login is unsuccessful (step S204: NO), the operation terminal 20 displays an error message indicating that the login is unsuccessful on a display (step S205).

In step S206, the operation terminal 20 specifies identification data that uniquely identifies the correspondence relationship between sensor data and personal information in response to an input operation of the inspector I. For example, the inspector I is preferably able to specify the identification information included in use history data in the screen on which use history data is displayed in a list as illustrated in FIG. 4.

Next, the operation terminal 20 outputs a display request of the sensor data to the data management apparatus 101 (step S207). The display request includes use history data (identification data, personal information).

Next, the data management apparatus 101 starts up the data display program PG2 in response to the display request from the operation terminal 20 (step S208) and searches the sensor data DB 102 for data by using the identification data as a key (step S209).

Next, in response to acquiring sensor data from the sensor data DB 102 (step S210), the data management apparatus 101 generates merge data obtained by associating the sensor data and the personal information with each other based on the identification data (step S211).

Next, in response to generating screen data including the merge data (step S212), the data management apparatus 101 transmits the screen data to the operation terminal 20 (step S213). Then, in response to receiving the screen data from the data management apparatus 101, the operation terminal 20 displays the merge data on a display (step S214), and the process ends.

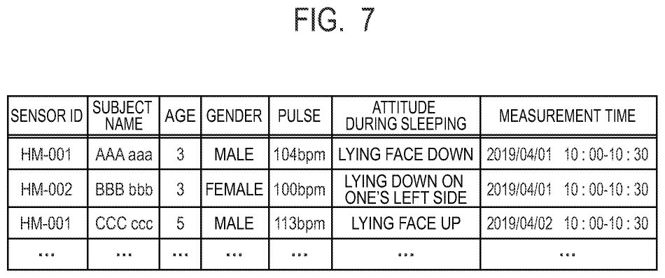

FIG. 7 is a diagram illustrating an example of merge data displayed on the operation terminal 20. Herein, data items of the sensor data may be a sensor ID, a subject name, an age, a gender, pulses, an attitude during sleeping, and measurement time. That is, the merge data illustrated in FIG. 7 is generated by associating the use history data including the personal information of FIG. 4 described above and the sensor data of FIG. 5 with each other based on identification data formed of a combination of a sensor ID and use time (measurement time). In the present example embodiment, a device that can display the merge data is limited to the operation terminal 20 that stores personal information that is in a correspondence relationship with the sensor data.

(C) Reference of Sample Data

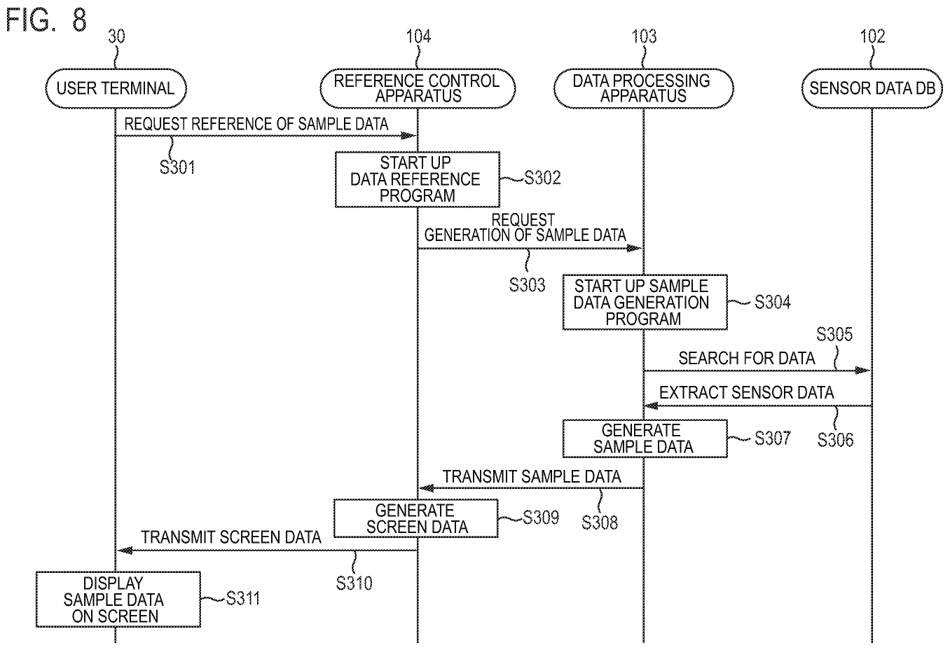

FIG. 8 is a sequence diagram illustrating an example of a reference process of sample data. The process is performed when the user U references sample data in which data format of the sensor data disclosed by the information processing system 1 is described as an example.

First, the user terminal 30 outputs a reference request of sample data to the reference control apparatus 104 (step S301). The reference control apparatus 104 starts up the data reference program PG5 in response to the reference request from the user terminal 30 (step S302).

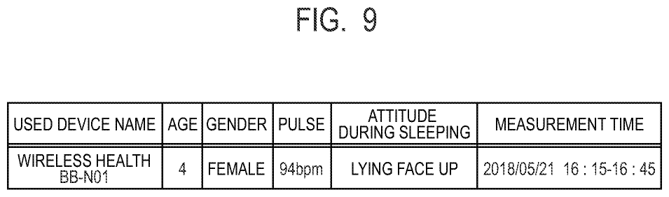

FIG. 9 is a diagram illustrating an example of sample data displayed on the user terminal 30. Herein, data items of the sample data may be a used device name, an age, a gender, pulses, an attitude during sleeping, and measurement time. The used device name is a model name of the sensor device 10 and differs from a sensor ID that is unique to the sensor device 10. The sample data is preferably data whose data value differs from the value of the original sensor data. For example, the sample data is generated by combining the sensor data illustrated in FIG. 7 or converting a part of data.

Next, the reference control apparatus 104 (data reference program PG5) outputs a generation request of the sample data to the data processing apparatus 103 (step S303).

Next, the data processing apparatus 103 starts up the sample data generation program PG4 (step S304). The data processing apparatus 103 (sample data generation program PG4) searches the sensor data DB 102 for data based on a specified item condition (step S305) and acquires a data value of the data item that matches the item condition from the sensor data DB 102 (step S306).

Next, the data processing apparatus 103 (sample data generation program PG4) generates sample data by combining the data values of the plurality of data items acquired from the sensor data DB 102 with each other at random (step S307). Next, the data processing apparatus 103 (sample data generation program PG4) transmits the sample data to the reference control apparatus 104 (step S308).

Next, in response to generating reference screen data of the sample data (step S309), the reference control apparatus 104 (data reference program PG5) transmits the screen data to the user terminal 30 that has output the reference request (step S310).

Then, in response to receiving the screen data from the reference control apparatus 104, the user terminal 30 displays the sample data on a screen (step S311), and the process ends.

(D) Reference of Sensor Data

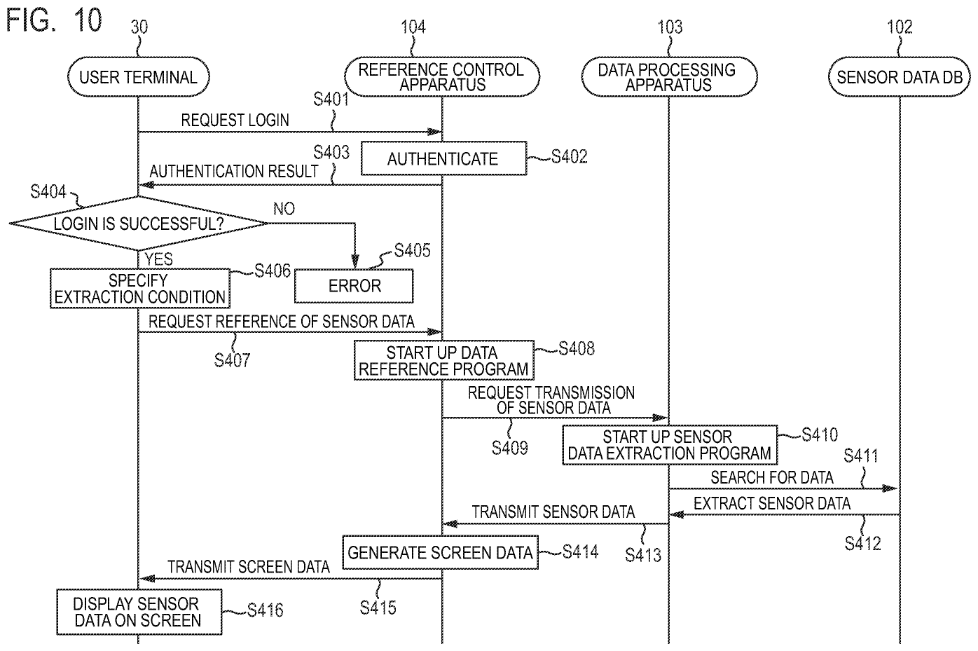

FIG. 10 is a sequence diagram illustrating an example of a reference process of sensor data. The process is performed when the user U references the sensor data in the information processing system 1. Note that, in the present example embodiment, as the reference condition of sensor data, it is required that the user U completed user registration in the information processing system 1 in advance.

First, the user terminal 30 outputs a login request to the reference control apparatus 104 based on the input operation of the user U (step S401). The login request includes authentication information such as a user ID and a password input by the user U.

Next, in response to performing an authentication process of the user U based on the authentication information included in the received login request (step S402), the reference control apparatus 104 transmits a result of the authentication to the user terminal 30 (step S403).

Next, the user terminal 30 determines whether or not the login is successful based on the authentication result in the reference control apparatus 104 (step S404). Herein, if the user terminal 30 determines that the login is successful (step S404: YES), the process proceeds to step S406.

On the other hand, if the user terminal 30 determines that the login is unsuccessful (step S404: NO), the user terminal 30 displays an error message indicating that the login is unsuccessful on a display (step S405).

In step S406, the user terminal 30 specifies an extraction condition of the sensor data based on the input operation of the user U. The extraction condition may be, for example, a data item of sensor data, types of sensor data, measurement time, or the like.

Next, the user terminal 30 outputs a reference request of the sensor data to the reference control apparatus 104 (step S407). The reference request includes the extraction condition specified in step S406.

Next, the reference control apparatus 104 starts up the data reference program PG5 in response to the reference request from the user terminal 30 (step S408).

Next, the reference control apparatus 104 (data reference program PG5) outputs a transmission request of the sensor data to the data processing apparatus 103 (step S409). The transmission request includes the extraction condition specified in step S406.

Next, the data processing apparatus 103 starts up the sensor data extraction program PG3 (step S410). The data processing apparatus 103 (sensor data extraction program PG3) searches the sensor data DB 102 for data based on the specified extraction condition (step S411) and acquires sensor data that matches the extraction condition from the sensor data DB 102 (step S412).

Next, the data processing apparatus 103 (sensor data extraction program PG3) transmits the sensor data to the reference control apparatus 104 (step S413).

Next, in response to generating reference screen data on the received sensor data (step S414), the reference control apparatus 104 (data reference program PG5) transmits the screen data to the user terminal 30 that has output the reference request (step S415).

Then, in response to receiving the screen data from the reference control apparatus 104, the user terminal 30 displays the sample data on the screen (step S416), and the process ends.

According to the information processing system 1 of the present example embodiment, there is an advantage that risk management related to personal information is no longer required when the acquired data is disclosed. This is because a third party has to acquire both of data including no personal information and data including personal information for identifying an individual from the disclosed data. According to the information processing system 1 of the present example embodiment, even when a third party makes unauthorized access to a database that stores data including no personal information, a database that stores personal information related to the corresponding data is unable to be identified. As described above, not only is the information processing system 1 less likely to be subjected to unauthorized access, but also personal information is not identified even in the event of data leak.

In response to collecting data (sensor data) including no personal information, the information processing system 1 of the present example embodiment stores the collected data in a storage device other than a device storing personal information. Since the data including no personal information is disclosed as it stands, the example embodiment can be implemented by using no specific algorithm or the like.

Further, an administrator or the like of the information processing system 1 is not required to perform additional work (for example, selection work of data to be disclosed, data processing work to prevent adverse effects on business by disclosing personal information, a procedure to obtain consent from the subject T before disclosure, or the like) or countermeasures for disclosing the collected data as with the conventional case. Moreover, a third party is able to freely reference the disclosed data. Thus, the information processing system 1 can contribute to the development of a technique or business by using the collected data.

In the information processing system 1 of the present example embodiment, the sensor data including no personal information is stored in the sensor data DB 102. When the inspector I uses the sensor data in the original use such as health management or the like of children, the data management apparatus 101 (data display program PG2) can separately acquire another database (storage unit 21 of the operation terminal 20) that stores personal information and data of the sensor data DB 102, dynamically associate the data with each other, and display and use the associated data.

Further, a third party (user U) is unable to directly access the sensor data stored in the sensor data DB 102 from the user terminal 30. However, the reference control apparatus 104 allows the user terminal 30 to reference the sensor data via a program referencing the sensor data DB 102 in response to the request from the user terminal 30. Accordingly, it is possible to avoid risk of leakage of personal information and widely disclose data to a third party who wants to reference the data.

For example, in a medical field or a working site such as a nursing service, childcare, or a school, there are many opportunities in which one can collect various information related to a person. Conventionally, however, since the collected data includes personal information, a use of the collected data is restricted. On the other hand, according to the information processing system 1 of the present example embodiment, it is also possible to widely disclose, to public, maintenance data or the like of infrastructure of a country or transportation facilities that has been unable to be externally disclosed so far. Facilitating use of data that has not been disclosed so far contributes to the development of various fields where data have been unable to be easily obtained and this has been an obstacle, and the worth of data is improved.

### Second Example Embodiment

An information processing system 2 in a second example embodiment will be described below. Note that references common to the references provided in the drawings of the first example embodiment refer to the same object. Thus, description of features common to those of the first example embodiment will be omitted, and different features will be described in detail.

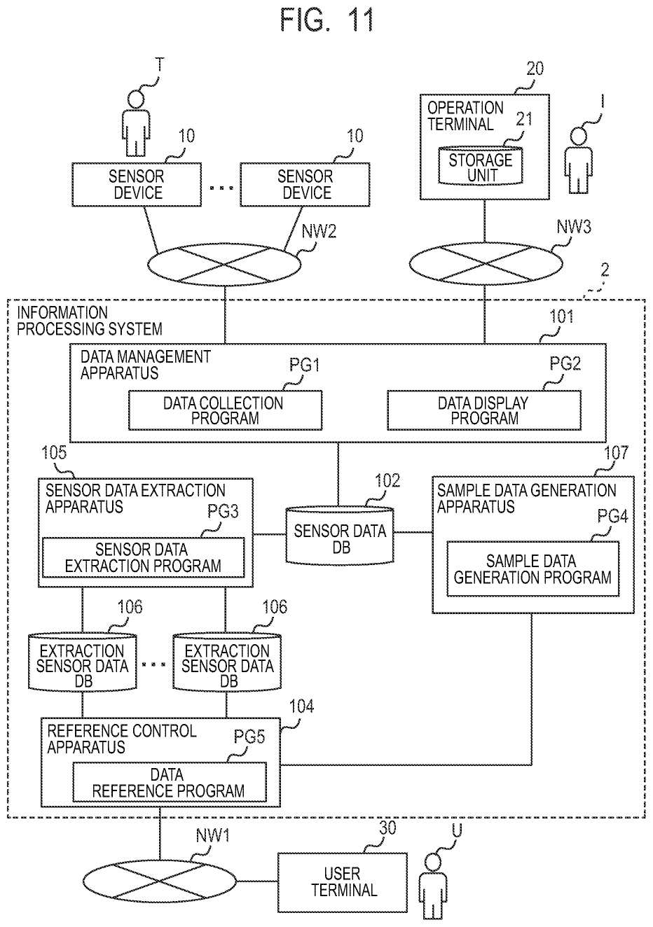

FIG. 11 is a block diagram illustrating an example of an overall configuration of the information processing system 2 in the present example embodiment. As illustrated in FIG. 11, the information processing system 2 in the present example embodiment is different from the information processing system 1 of the first example embodiment in that the sensor data extraction apparatus 105, the plurality of extraction sensor data DBs 106, and the sample data generation apparatus 107 are further included. Each apparatus is a server computer, for example.

In the present example embodiment, the sensor data DB 102 is connected to the data management apparatus 101, the sensor data extraction apparatus 105, and the sample data generation apparatus 107. Further, the reference control apparatus 104 is connected to the extraction sensor data DB 106, the sample data generation apparatus 107, and the user terminal 30 but is not directly connected to the sensor data extraction apparatus 105 and the sensor data DB 102. That is, the reference control apparatus 104 is unable to directly reference the sensor data DB 102.

The sensor data extraction apparatus 105 has the sensor data extraction program PG3. The sensor data extraction program PG3 of the present example embodiment is different from that of the first example embodiment in that the sensor data extracted from the sensor data DB 102 is classified based on a predetermined classification condition and registered in any of the plurality of extraction sensor data DBs 106.

The extraction sensor data DB 106 stores sensor data classified and extracted by the sensor data extraction program PG3, respectively. For example, multiple types of sensor data that are respectively measured about a certain subject T may be stored in different extraction sensor data DBs 106 separately for each type of sensor data. The type of sensor data is defined in accordance with the number or the content of data items forming the sensor data, for example.

The sample data generation apparatus 107 has the sample data generation program PG4. That is, two functions of the data processing apparatus 103 of the above first example embodiment are separately implemented in the sensor data extraction apparatus 105 and the sample data generation apparatus 107.

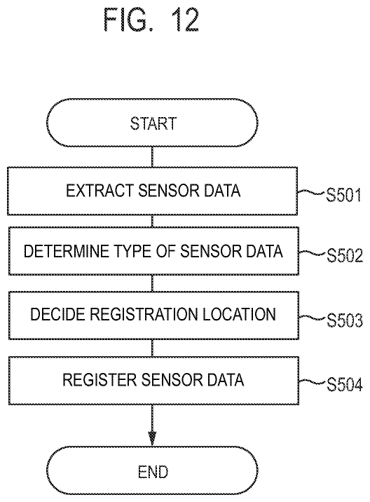

FIG. 12 is a flowchart illustrating an example of a classification process of sensor data. The process is performed by the sensor data extraction apparatus 105 (sensor data extraction program PG3) at a predetermined cycle, for example.

First, in response to extracting sensor data from the sensor data DB 102 (step S501), the sensor data extraction apparatus 105 determines the type of the sensor data (step S502). The type of the sensor data is determined by the type of data item forming sensor data, the type of sensor device 10, or the like, for example.

Next, the sensor data extraction apparatus 105 determines a registration location of sensor data in accordance with the type of sensor data (step S503). The sensor data extraction apparatus 105 then registers the sensor data in the extraction sensor data DB 106 of the registration location (step S504).

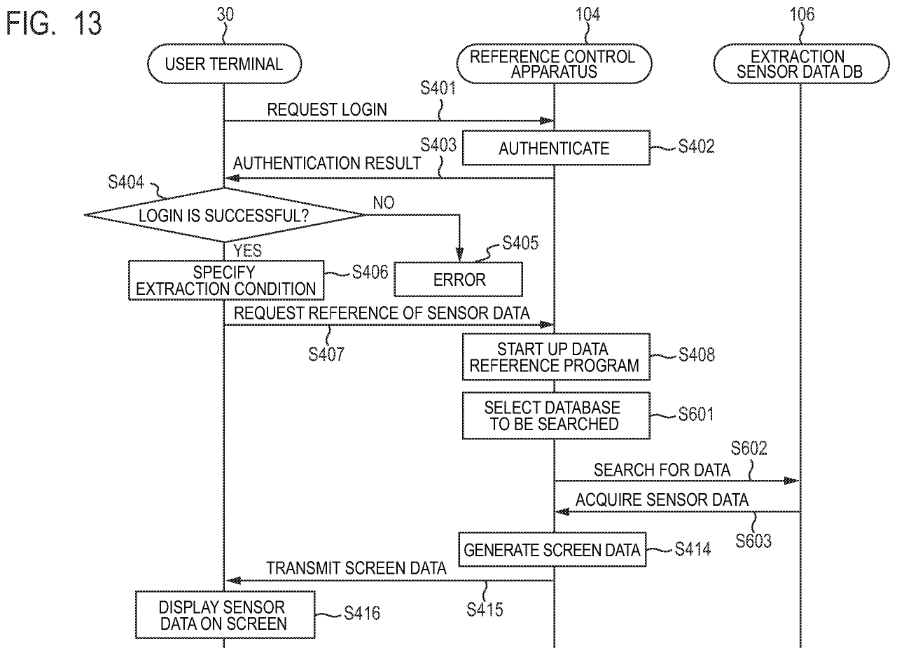

FIG. 13 is a sequence diagram illustrating an example of a reference process of sensor data. The process is performed when the user U requests reference of sensor data disclosed by the information processing system 2. Note that, since the processes of step S401 to step S408 and step S414 to step S416 are the same as those of FIG. 10 described above, only the processes performed between step S408 and step S414 will be described below.

In step S601, the reference control apparatus 104 selects a database to be searched based on the extraction condition specified in step S406. Specifically, the database storing the data item specified by the extraction condition is selected out of the plurality of extraction sensor data DBs 106.

In step S602, the reference control apparatus 104 searches the extraction sensor data DB 106 selected in step S601 for data. The reference control apparatus 104 then acquires the sensor data that matches the extraction condition from the extraction sensor data DB 106 (step S603). The process then proceeds to step S414.

The reference control apparatus 104 acquires the sensor data from the specific extraction sensor data DB 106 storing data on a necessary data item in response to the request from the user terminal 30 and causes the user terminal 30 to reference the data.

According to the information processing system 2 of the present example embodiment, it is difficult from the user terminal 30 side to access the operation terminal 20 storing personal information or the sensor data DB 102 centrally storing the sensor data. Further, even when data leakage from the extraction sensor data DB 106 is caused by unauthorized access to the reference control apparatus 104, a third party is unable to identify what value is represented by the data. As described above, not only is the information processing system 1 less likely to be subjected to unauthorized access, but also personal information is not identified even in the event of data leak.

### Third Example Embodiment

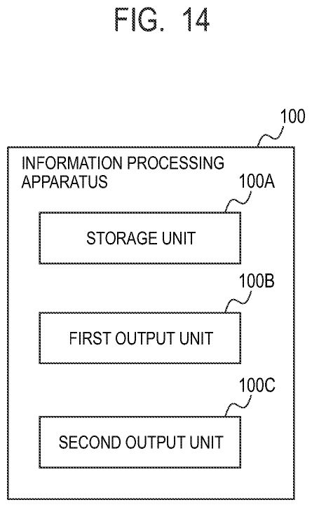

FIG. 14 is a block diagram illustrating an example of an overall configuration of an information processing apparatus 100 in the present example embodiment. The information processing apparatus 100 has a storage unit 100A, a first output unit 100B, and a second output unit 100C. The storage unit 100A stores first data including no personal information on a person and does not store second data including personal information. The first output unit 100B outputs the first data of the storage unit 100A to a first apparatus in response a request from the first apparatus. The second output unit 100C acquires the second data including personal information from a second apparatus and outputs, to the second apparatus, third data obtained by combining the personal information with the first data.

According to the present example embodiment, the information processing apparatus 100 that can prevent leakage of personal information and facilitate use of data including no personal information is provided.

### Modified Example Embodiments

The present invention is not limited to the example embodiments described above and can be changed as appropriate within the scope not departing from the spirit of the present invention.

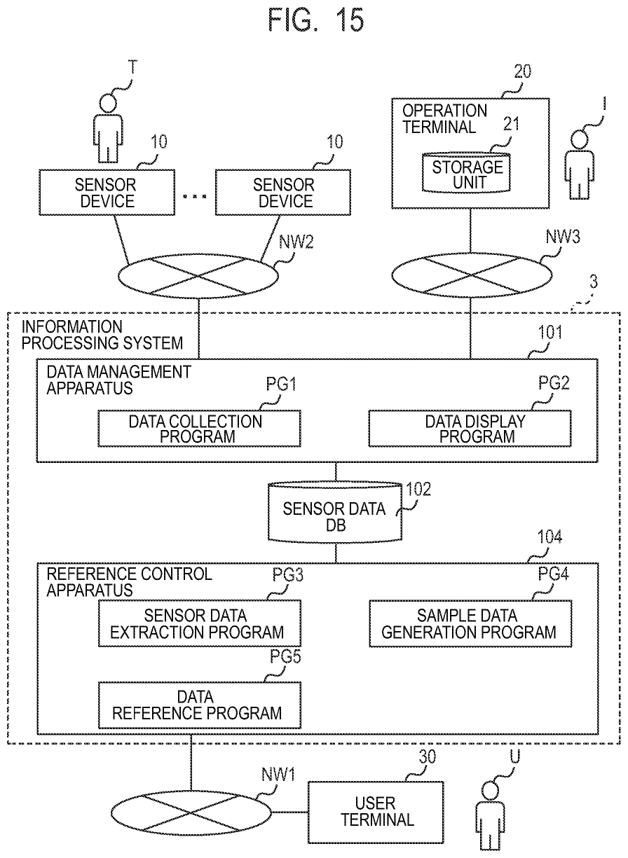

FIG. 15 is a block diagram illustrating an example of an overall configuration of an information processing system 3 in the modified example embodiment. The present example embodiment describes a configuration taking into consideration of two points that devices storing data are not directly connected to each other and that there is risk of unauthorized access from the external. Herein, a configuration in which all of the respective programs included in the data processing apparatus 103 and the reference control apparatus 104, respectively, in the above first example embodiment are included in the reference control apparatus 104 is illustrated.

The flow regarding the inspector I who collects data is the same as that of the first example embodiment. The user U (third party) who references data accesses the reference control apparatus 104 for confirming whether or not there is data that the user U want to reference from the user terminal 30. The user U confirms whether or not there is data that the user U wants to reference by using sample data generated by the reference control apparatus 104, and when there is data that the user U wants to reference, the user U outputs a reference request to the reference control apparatus 104. The reference control apparatus 104 holds data to be referenced by the user U as temporary volatile data, and the data may be discarded when the access from the user terminal 30 is completed.

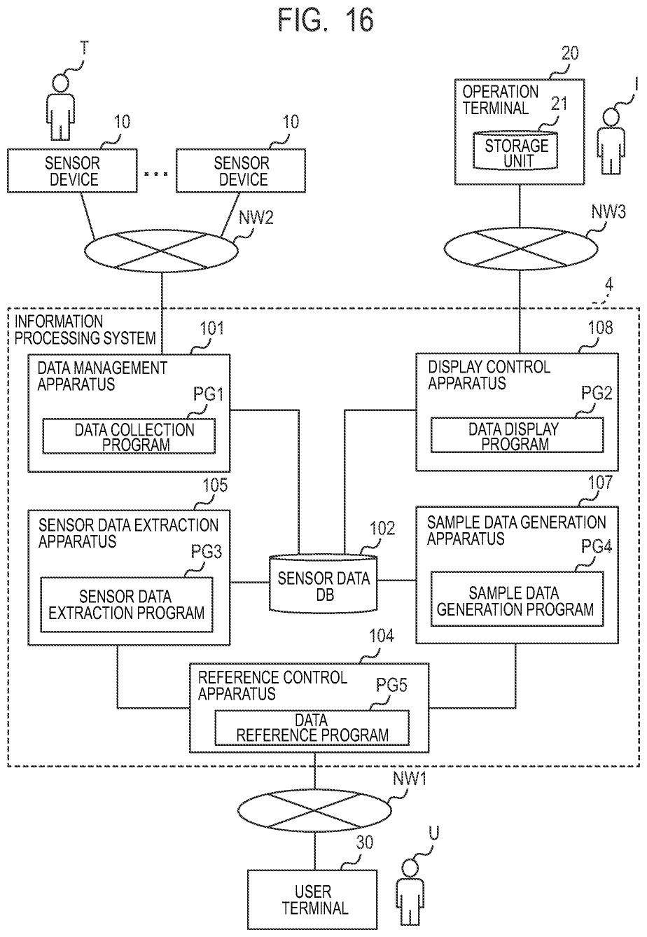

FIG. 16 is a block diagram illustrating an example of an overall configuration of an information processing system in a modified example embodiment. In the information processing system 4 of the present example embodiment, locations where the data collection program PG1 and the data display program PG2 are implemented are separated into the data management apparatus 101 and the display control apparatus 108. Further, locations where the sensor data extraction program PG3 and the sample data generation program PG4 are implemented are separated into the sensor data extraction apparatus 105 and the sample data generation apparatus 107.

Moreover, installation places of the respective apparatuses forming the system may not be the same. For example, the data management apparatus 101, the sensor data DB 102, the data processing apparatus 103, and the reference control apparatus 104 may be installed in a single place, and only the sensor data DB 102 may be installed in another place.

Further, in the above example embodiments, the configuration in which the operation terminal 20 and the sensor device 10 are not directly connected to each other has been described. In such a case, even when the operation terminal 20 and the sensor device 10 are not directly connected to each other, the operation terminal 20 may be configured to be able to easily acquire identification data. For example, when the sensor device 10 has a function of displaying identification data on a screen by using a barcode or the like, the identification data can be acquired by reading the barcode in the operation terminal 20, and personal information input by the inspector I and the identification data can be stored in the storage unit 21 in association with each other.

The scope of each of the example embodiments further includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the program itself. Further, one or two or more components included in the example embodiments described above may be a circuit such as an application specific integrated circuit (ASIC), a field programmable gate array (FPGA), or the like configured to implement the function of each component.

As the storage medium, for example, a floppy (registered trademark) disk, a hard disk, an optical disk, a magneto-optical disk, a Compact Disk (CD)-ROM, a magnetic tape, a nonvolatile memory card, or a ROM can be used. Further, the scope of each of the example embodiments includes an example that operates on Operating System (OS) to perform a process in cooperation with another software or a function of an add-in board without being limited to an example that performs a process by an individual program stored in the storage medium.

Further, a service implemented by the function of each of the example embodiments described above may be provided to a user in a form of Software as a Service (SaaS).

Note that all the example embodiments described above are mere examples of example embodiment in implementing the present invention, and the technical scope of the present invention should not be construed in a limiting sense by these example embodiments. That is, the present invention can be implemented in various forms without departing from the technical concept thereof or the primary feature thereof.

The whole or part of the example embodiments disclosed above can be described as, but not limited to, the following supplementary notes.

### (Supplementary Note 1)

An information processing system comprising:

a storage unit that stores first data including no personal information on a person and does not store second data including the personal information;

a first output unit that outputs the first data to a first apparatus in response to a request from the first apparatus; and

a second output unit that acquires the second data from a second apparatus and outputs, to the second apparatus, third data obtained by combining the personal information with the first data.

### (Supplementary Note 2)

The information processing system according to supplementary note 1, wherein the second data includes identification data that uniquely identifies a correspondence relationship between the personal information and the first data.

### (Supplementary Note 3)

The information processing system according to supplementary note 2,

wherein the first data includes a plurality of data items, and

wherein the identification data includes at least two of the plurality of data items included in the first data.

### (Supplementary Note 4)

The information processing system according to supplementary note 3 further comprising a third output unit that generates and outputs fourth data whose data value is different from a data value of the first data to the first apparatus in response to a request from the first apparatus.

### (Supplementary Note 5)

The information processing system according to supplementary note 2 or 3, wherein the identification data includes at least an identifier of a device that acquired the first data from the person and acquisition time and date of the first data.

### (Supplementary Note 6)

The information processing system according to any one of supplementary notes 1 to 5,

wherein the storage unit is provided in a first server, the first output unit is provided in a second server, and the second output unit is provided in a third server,

wherein the first server is configured to communicate with the second server and the third server, respectively, and

wherein communication between the second server and the third server is restricted.

### (Supplementary Note 7)

The information processing system according to any one of supplementary notes 1 to 6,

wherein the storage unit includes a plurality of storage devices that classify and store the first data by each type of the first data, and

wherein the first output unit acquires the first data from a storage device corresponding to the type specified by the first apparatus and outputs the acquired first data to the first apparatus.

### (Supplementary Note 8)

An information processing method performed by an information processing system including a storage device that stores first data including no personal information on a person and does not store second data including the personal information, the information processing method comprising:

outputting the first data to a first apparatus in response to a request from the first apparatus; and

acquiring the second data from a second apparatus and outputting, to the second apparatus, third data obtained by combining the personal information with the first data.

### (Supplementary Note 9)

The information processing method according to supplementary note 8, wherein the second data includes identification data that uniquely identifies a correspondence relationship between the personal information and the first data.

### (Supplementary Note 10)

A program that causes a computer having a storage device that stores first data including no personal information on a person and does not store second data including the personal information to perform:

outputting the first data to a first apparatus in response to a request from the first apparatus; and

acquiring the second data from a second apparatus and outputting, to the second apparatus, third data obtained by combining the personal information with the first data.

What is claimed is:

1. An information processing system comprising:
a storage unit that stores first data including no personal information on a person and does not store second data including the personal information;
a first output unit that outputs the first data to a first apparatus in response to a request from the first apparatus; and
a second output unit that acquires the second data from a second apparatus and outputs, to the second apparatus, third data obtained by combining the personal information with the first data.

2. The information processing system according to claim 1, wherein the second data includes identification data that uniquely identifies a correspondence relationship between the personal information and the first data.

3. The information processing system according to claim 2,
wherein the first data includes a plurality of data items, and
wherein the identification data includes at least two of the plurality of data items included in the first data.

4. The information processing system according to claim 3 further comprising a third output unit that generates and outputs fourth data whose data value is different from a data value of the first data to the first apparatus in response to a request from the first apparatus.

5. The information processing system according to claim 2, wherein the identification data includes at least an identifier of a device that acquired the first data from the person and acquisition time and date of the first data.

6. The information processing system according to claim 1,
wherein the storage unit is provided in a first server, the first output unit is provided in a second server, and the second output unit is provided in a third server,
wherein the first server is configured to communicate with the second server and the third server, respectively, and
wherein communication between the second server and the third server is restricted.

7. The information processing system according to claim 1,
wherein the storage unit includes a plurality of storage devices that classify and store the first data by each type of the first data, and
wherein the first output unit acquires the first data from a storage device corresponding to the type specified by the first apparatus and outputs the acquired first data to the first apparatus.

8. An information processing method performed by an information processing system including a storage device that stores first data including no personal information on a person and does not store second data including the personal information, the information processing method comprising steps of:
outputting the first data to a first apparatus in response to a request from the first apparatus; and
acquiring the second data from a second apparatus and outputting, to the second apparatus, third data obtained by combining the personal information with the first data.

9. The information processing method according to claim 8, wherein the second data includes identification data that uniquely identifies a correspondence relationship between the personal information and the first data.

10. A non-transitory storage medium in which a program is stored, the program that causes a computer having a storage device that stores first data including no personal information on a person and does not store second data including the personal information to perform:
outputting the first data to a first apparatus in response to a request from the first apparatus; and
acquiring the second data from a second apparatus and outputting, to the second apparatus, third data obtained by combining the personal information with the first data.
